(19) United States
(12) Patent Application Publication
(10) Pub. No.: US 2025/0352238 A1
(43) Pub. Date: Nov. 20, 2025

(54) PROTECTION SYSTEM FOR MEDICAL PROCEDURES

(71) Applicant: MIMIR'S Well-ness LLC, Reno, NV (US)

(72) Inventors: Bridget Martinez, Reno, NV (US); Donald Mario Robert Harker, Reno, NV (US); Tony Duy Le, Lebanon, TN (US)

(73) Assignee: MIMIR'S Well-ness LLC, Reno, NV (US)

(21) Appl. No.: 19/281,308

(22) Filed: Jul. 25, 2025

Related U.S. Application Data
(63) Continuation-in-part of application No. PCT/US24/12456, filed on Jan. 22, 2024.
(60) Provisional application No. 63/441,604, filed on Jan. 27, 2023.
(60) Provisional application No. 63/703,711, filed on Oct. 4, 2024.

(51) Int. Cl. A61B 17/326; A61B 17/00

(52) U.S. Cl. CPC A61B 17/326; A61B 2017/00477

(57) ABSTRACT

An apparatus for use during a medical procedure with a medical device on or proximate to a penis comprises a body with an opening therethrough sized at a minimum that an infant penis can project through the opening in use during the medical procedure. During the procedure the apparatus is placed over a patent's penis with the penis projecting through the opening and with the body proximate to the patient's skin for providing protection during the medical procedure. An attachment is present on the inner diameter of the mesh enabling attachments to currently used medical devices in circumcisions, such as Gompco clamps, providing optimal surface area protection.

### CROSS-REFERENCE TO RELATED APPLICATION(S)

This application is a continuation-in-part of PCT Application No. PCT/US24/12456 filed on Jan. 22, 2024, which claims the benefit of and priority to provisional application Ser. No. 63/441,604 filed on Jan. 27, 2023. This application also claims the benefit of and priority to provisional application 63/703,711 filed on Oct. 4, 2024. All of these applications are incorporated in their entirety herein by reference.

### BACKGROUND

Medical procedures on a penis include penis enlargement, circumcision, implantation in a penis, fix a bent penis (penile plication), fix penis fracture, vasectomy, vasectomy reversal, prostate biopsy, urolift, transurethral needle ablation, prostate procedures such as transurethral resection of the prostate (TURP) and transurethral incision of the prostate (TUIP), central line placement, and orchiopexy.

Circumcision of newborn males is common at least in the United States. A risk in these medical procedures, and particularly circumcision, is unintentionally injuring the patient from accidental slip of the surgical blade while cutting the foreskin; Those injuries can be severe, and include cutting through the skin, into vital tissues, nicking or cutting a femoral artery. Accordingly, there is a need for an apparatus and a method that provides protection for a patient during a medical procedure on or proximate to a penis. Documents of interest are CN 202682170; CN21096269, U.S. Pat. No. 6,580,011, and US Publication No. 2012/0220912 and PCT/US24/12437.

### SUMMARY

The present invention is directed to a system that provides protection to a patient during a medical procedure such as circumcision, the system comprising an apparatus and a method for using the apparatus. An attachment is present on the inner diameter of the mesh enabling attachments to currently used medical devices in circumcisions, such as Gompco clamps, providing optimal surface area protection.

The protection apparatus comprises a body, preferably a mesh, having an outer perimeter and a circular or oval opening therethrough, the opening having an edge. The opening is sized to comfortably fit a penis, and preferably has a diameter of from about 1 to about 1.5 inch. The outer perimeter has a diameter of from about 6 to about 10 inches.

Preferably the mesh is made of wires about 0.5 to about 1.5 millimeters in diameter. The wires of the mesh are sufficiently close together that a scalpel pushed with a force of 20 Newtons cannot penetrate the mesh. Typically, the mesh is sufficiently flexible that the mesh can be doubled on itself.

Preferably the opening and the perimeter are both circular.

Preferably the edges of the opening and the perimeter are sufficiently smooth to not injure or irritate a patient during circumcision. However, for rough edges, a covering can be on the perimeter of the body, or the edge of the opening, or both.

In a method according to the present invention, the following steps can be performed:
(a) selecting an apparatus comprising a body having an opening therethrough, the opening being at or near the center of the body;
(b) placing the selected apparatus over the patient's penis with the penis projecting through the opening and with the mesh proximate to the patient's skin; and
(c) after step (b) circumcising the projecting penis.

Stated another way, the apparatus is useful during a medical procedure proximate to or involving a penis and comprises a flexible body having an outer perimeter with an opening therethrough, the opening having a diameter of from about 0.2 to about 1.5 inch. The outer perimeter of the body has a diameter of at least about 6 inches. The body is sufficiently strong that a No. 10 scalpel pushed with a force of 10 Newtons, preferably with a force of 20 Newtons, more preferably with a force of 15 Newtons, cannot penetrate the body. Since medical procedures can involve a needle, preferably the body is sufficiently strong that a 28-gauge needle pushed with a force of 10 Newtons cannot penetrate the body, and more preferably 28-gauge needle pushed with a force of 20 Newtons cannot penetrate the body, and most preferably the body is sufficiently strong that a 28-gauge needle pushed with a force of 15 Newtons cannot penetrate the body. It is desirable that the body is sufficiently strong that both the scalpel and needle, cannot penetrate, although it can be sufficient that the body resists such a force for either the scalpel or needle.

Size opening can depend on a particular application. Generally, the opening is sufficiently large that an infant penis having a circumference of 0.5 inch can project through the opening. The opening is typically centrally or near centrally located in the body. Preferably the body is a mesh. The mesh can have an aperture size of from about 0.02 mm to about 0.2 mm, and preferably a maximum of about 0.16 mm. The mesh can be formed of wires having a diameter of from about 0.02 mm to about 0.15 mm, and preferably a maximum of about 0.12 mm.

The mesh can be made of stainless steel with square shaped apertures of about 0.14 mm per side, a wire diameter of about 0.112 mm, 100 mesh per inch and open area between wires of about 30%, and a weight of about 0.6 Kg per square meter.

In a typical medical procedure on a patient the following steps are performed:
A. select an apparatus comprising a flexible body having an opening therethrough sized at a minimum that an infant penis having a circumference of 0.5 inch can project through the opening;
B. placing the selected apparatus over the patient's penis with the penis projecting through the opening and with the body proximate to the patient's skin; and
C. after step B performing the medical procedure such as a circumcision.

In an alternate version of the present invention the apparatus includes an interior mesh ring, the interior mesh ring having a first diameter and a second diameter, wherein the first diameter is equal to or greater than the diameter of the opening and the second diameter is less than the diameter of the first diameter; and an attachment for removably securing the apparatus to a medical device, such as a circumcision device.

In a preferred version the apparatus attachment is a fastener at the opening and the circumcision device is a circumcision clamp. Preferably, the opening is centrally or near centrally located, and has a diameter of from about 1 to about 1.5 inch. The outer perimeter of the mesh has a diameter of about 6 to about 10 inches and the mesh being made of wires sufficiently close together that a scalpel pushed with a force of 20 Newtons cannot penetrate the mesh.

Preferably, the edges of the interior mesh ring opening and the perimeter are sufficiently smooth to not injure or irritate a patient during circumcision.

The attachment can have an outer perimeter which is circular in cross section, matching the configuration of a circumcision clamp. The apparatus can be attached to the circumcision device by the attachment. Preferably, the body of the apparatus is sufficiently strong that a number 10 scalpel pushed with a force of 10 Newtons cannot penetrate the body and most preferably, the body is sufficiently strong that a 28 gauge needle pushed with a force of 10 Newtons cannot penetrate the body.

The present invention can comprise a kit comprising the apparatus and a circumcision clamp.

In another version, a method for performing a medical procedure using a medical device on a patient comprising the steps of:
a) selecting an apparatus comprising a flexible body having an opening therethrough sized at a minimum that an infant penis having a circumference of 0.5 inch can project through the opening; an interior mesh ring having a first diameter and a second diameter, wherein the first diameter is equal to or greater than the diameter of the opening and the second diameter is less than the first diameter; and, an attachment for attaching the apparatus to the medical device;
b) placing a drape over the abdominal area of the patient including the femoral arteries;
c) placing the selected apparatus over the drape and over the patient's penis with the penis projecting through the opening and the interior mesh ring and with the body proximate to the patient's skin for protecting the patient's abdominal area from penetration during the medical procedure;
d) attaching the selected apparatus to the medical device with the attachment; and,
e) after step (d) performing the medical procedure.

The medical device in the medical procedure can be a circumcision clamp and the attachment is a clamp and this method is for performing a circumcision.

In a preferred embodiment the body of the apparatus is a mesh.

In a preferred version the apparatus further comprises an attachment for removably securing the apparatus to the medical device, and in a most preferred embodiment the medical device is a circumcision clamp.

### DRAWINGS

These and other features, aspects, and advantages of the present invention will become better understood with reference to the following drawings, wherein:

FIG. 1 is a top plan view of an apparatus according to the present invention;

FIG. 2 is a top plan view of an apparatus according to the present invention that includes a covering on the periphery of the mesh and the edge of the penis opening;

FIG. 3 is a schematic view showing use of an apparatus according to the present invention.

FIG. 4A is a side view of an apparatus according to the present invention that includes an internal mesh ring and an attachment;

FIG. 4B is a side view of the apparatus shown in 4A with the internal mesh ring and attachment connected;

FIG. 5 is a side exploded view of the apparatus shown in FIG. 4 with a medical device which is a circumcision clamp in this figure;

FIG. 6 is a perspective view of the apparatus shown in FIG. 4 and FIG. 5 receiving the medical device or circumcision clamp;

FIG. 7 is top perspective view showing an alternative shape of the attachment, a semi-circle with a circumcision device attached;

FIG. 8A is a side view of an apparatus with the shape of the attachment being configured to match the shape of the clamp; and

FIG. 8B is a top plan view of the attachment and medical device shown in FIG. 8A.

### DESCRIPTION

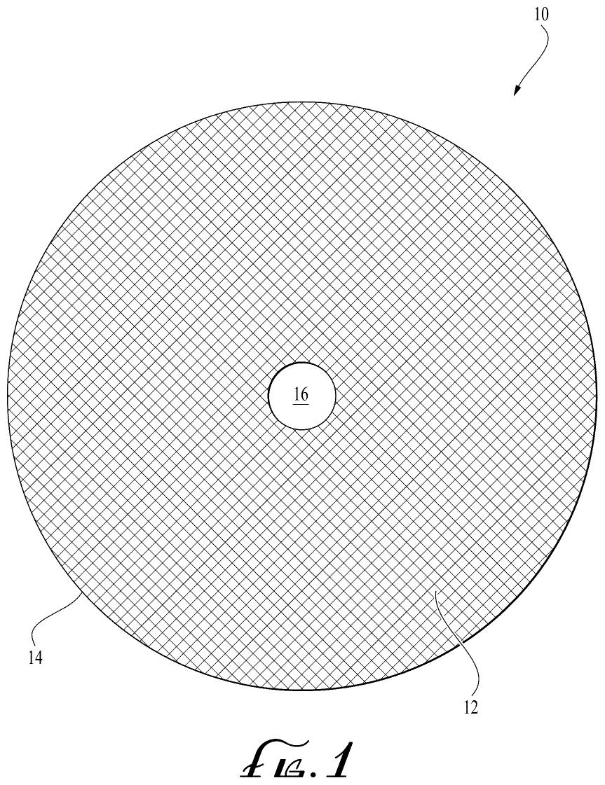

With regard to FIG. 1 an apparatus 10 useful during circumcision comprises a body 12, which can also be referred to as a substrate, having an outer perimeter 14 and an opening 16 therethrough. Preferably the opening is centrally located; thus, the apparatus is doughnut-shaped.

The perimeter has a diameter of at least about 6 inches, and typically from about 6 to about 10 inches to provide adequate coverage in the method described below. The opening 16 can be centrally located, but also be off center such as near centrally located.

In FIG. 1 the perimeter 14 is shown as being circular, but the apparatus is useful with any shaped perimeter such as rectangular, polygonal, oval and triangular.

The opening 16 is sized to fit an infant's penis projecting through the opening 16. An infant's penis is typically 0.5 inch in circumference. A preferred diameter for the opening is from about 1 to about 1.5 inch.

Although it is preferred that the opening 16 be circular, it can be in other shapes such as oval or triangular. When referring to the “diameter” of the opening 16 when it is not circular, reference is being made to the shortest distance across the opening. Thus, if an opening is oval in shape, then the term “diameter” refers to the small diameter of the oval. Stated in another way, the opening 16 in all versions needs to be sufficiently large that the infant's penis can extend therethrough without difficulty.

The body 12 is formed from a material that is flexible and not irritating against a patient's body, sufficiently resistant to penetration that a scalpel or needle pushed with a force of 10 Newtons, preferably 20 Newtons, and more preferably 50 Newtons, cannot penetrate the body, and sterilizable, such as by an autoclave. Stated another way, the body is not to be puncturable by a needle or scissors and scalpel when the medical professional uses a reasonable amount of force, which typically is at least 20 Newtons, and usually 50 Newtons, during the procedure so an accidental slip of blade or needle or scissors during a circumcision procedure does not occur. So there is no penetration by a force of 10 Newtons or less, preferably 20 Newtons or less, and more preferably no penetration by a force of 50 Newtons or less. Of course, the body can be designed that forces even higher than 50 Newtons do not result in penetration.

To determine if a material is suitable by testing with a scalpel, this can be determined by using a scalpel normally used by a surgeon, namely a number 10 scalpel according to ASTM F1790/F1790M-15 effective the date of filing of this application. To determine if a material is suitable with a needle typically used by a surgeon, that is determined with a 28 gauge needle according to ASTM F2878-19 effective the date of filing of this application. So, the forces stated above are determine with a number 10 scalpel and 28 gauge needle utilizing these ASTM tests. The material preferably satisfies these tests for both a needle and scalpel, but in some instances, depending on the medical procedure, the material needs to be satisfactory for just the needle or the scalpel.

Preferably the material is a mesh, and more particularly stainless-steel mesh such as a mesh available under the brand name Labalpha, particularly stainless steel 304, smooth finish, 100 mesh, aperture of about 0.14 millimeter, and a wire diameter of about 0.11 millimeter. This mesh resisted penetration by a force in excess of 130 Newtons.

Other materials that can be suitable include plastics such as plastic polymers including, polycarbonate, polypropylene, polyethylene, polyvinylchloride, acrylonitrile butadiene styrene, polystyrene, polyethylene terephthalate glycol, and polymethyl methacrylate. Preferably the material used is medical grade.

A suitable mesh is made of stainless steel with a squared shaped apertures of 0.14 mm per side and wire diameter of 0.112 mm, circular with a diameter of about 9 inches and a circular central aperture of about 1.5 inches in diameter, with an open area (between wires) of about 30%.

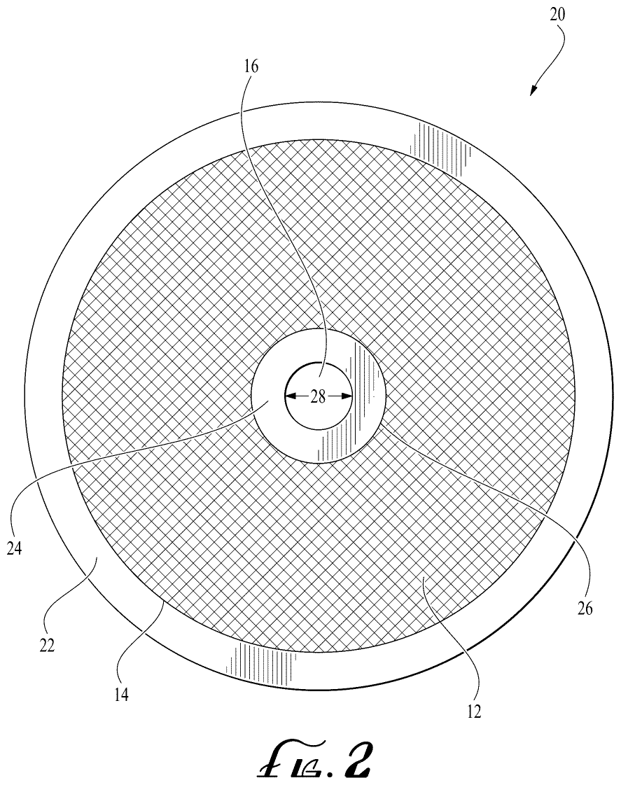

With reference to FIG. 2, an alternate version of an apparatus 20 according to the present invention is shown. The apparatus of FIG. 2 is an adaptation of the apparatus 10 of FIG. 1. The version of FIG. 2 is adapted for situations where the periphery 14 of the body of FIG. 1 is not sufficiently smooth to avoid injury or irritation to a patient during circumcision. Accordingly, a covering 22 can be provided on the periphery 14 of the body. Likewise, a covering 24 can be placed on the edge 26 on the central opening.

The dimensions that are given for the diameter of the opening 16 are based on inclusion of the covering so that in FIG. 2 the diameter is shown by double arrow 28. Likewise, the diameter of the apparatus of about 6 to about 10 inches includes the covering 22.

Either or both coverings 22 or 24 can be used. The coverings 22 and 24 are preferably formed of a durable material such as medical grade silicone, polyethylene, polyvinylchloride, polypropylene, or Kevlar. The coverings can be formed as separate pieces that are attached to the body such as by epoxy adhesive or crimping. Alternatively, the covering can be applied as a coating, such as a spray-on coating that hardens in place.

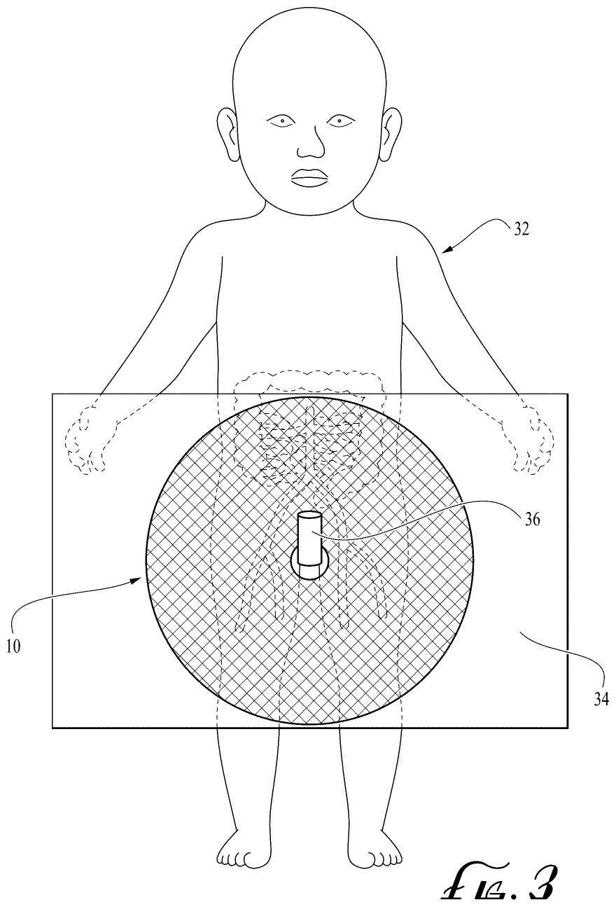

With reference to FIG. 3, circumcision is conducted as normal, but with the addition of the safety apparatus 10. As shown schematically in FIG. 3, an infant 32 is laid on his back and a sterile drape 34 is placed over the infant covering the abdominal area, including the femoral arteries. The apparatus 10 is placed over the drape proximate to the infant's skin with the infant's penis 36 projecting through the opening 16. Generally, the apparatus is placed against the sterile drape. A conventional circumcision is then performed on the projected penis. Circumcision is typically performed by placing a clamp 37, which can be the medical device in a circumcision procedure, on the penis. Typical clamps 37 used are the Gomco clamp, the Plastibell device, and the Mogen clamp. The clamp 37, ring or bell is attached to the penis and then the medical professional clips off excess foreskin.

Although the apparatus and method are particularly useful with infants, they can be used on a male of any age. Also there may be applications for a female such as central line placement.

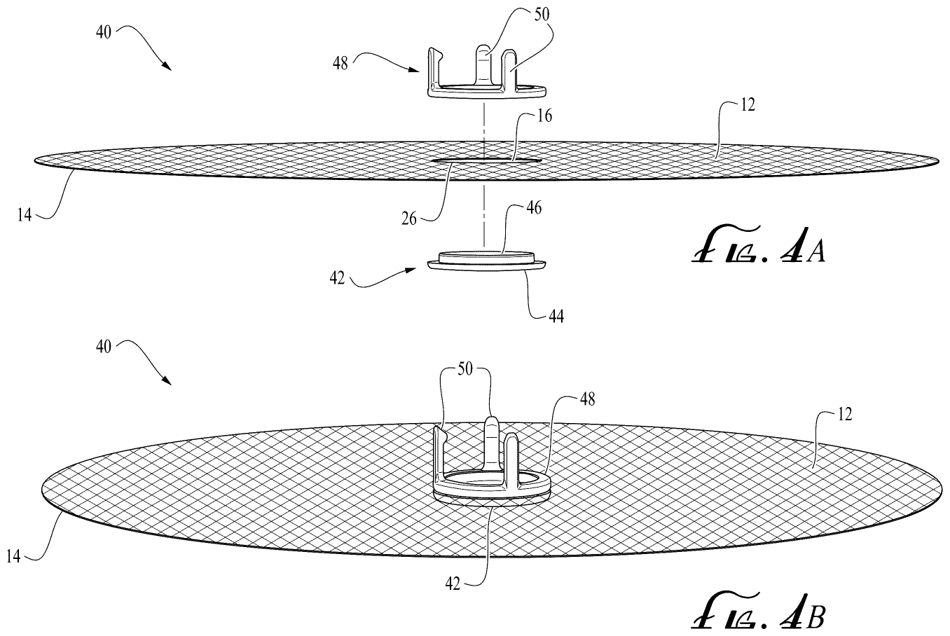

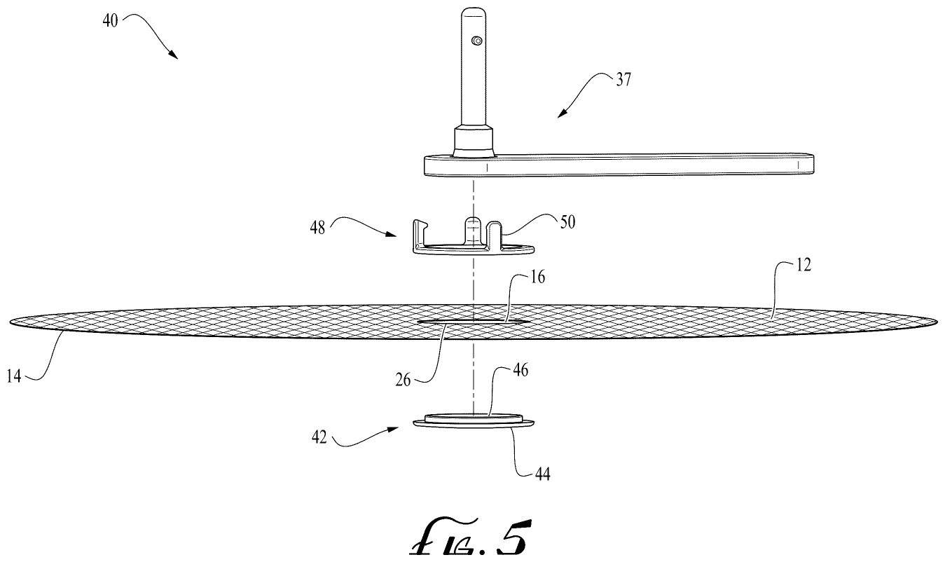

Referring now to FIG. 4, another version of an apparatus 40 according to the present invention is shown. The apparatus 40 of FIG. 4 is an adaptation of the apparatus 10 from FIG. 1 or the apparatus 20 from FIG. 2. The version in FIG. 4 is adapted with an internal mesh ring 42. The internal mesh ring 42 surrounds the edge 26 of the central opening 16. The interior mesh ring 42 has a first diameter 44 and a second diameter 46. As shown, the first diameter 44 is equal to or greater than the diameter 28 of the opening 16 and the second diameter is less than the first diameter 44. In a preferred version the second diameter is less than the diameter of 28 of the opening and the first diameter is greater than the diameter of the opening 16. When the first diameter 44 is greater than the opening 16 it prevents the internal mesh ring 42 from coming through the opening 16. When the second 46 diameter is less than the diameter of the opening 16, this allows a portion of the internal mesh ring 42 to extend through the opening 16. The internal surface of internal mesh ring 42 surface is similar to the covering 24 in FIG. 2 in that the internal mesh ring 42 is preferably smooth to protect the patient from injury. Depending on the configuration the internal mesh ring 42 can also be used to assist in securely affixing the body 12 to the patient.

The internal mesh ring 42 can also be used in connection with an attachment 48. The attachment 48 is used to removably secure the apparatus 40 to the circumcision device 37. The internal mesh ring 42 and the attachment 48 are preferably made with a semirigid plastic such as polypropylene, polyethylene, polyurethane, or polycarbonate, preferably medical grade. The mesh ring 42 and 48 can be manufactured together as a single piece or separately as two pieces, and are preferably made in three sizes varying from 1.2 inches to about 1.4 inches for the exterior diameter of the ring. The sizes are intended to receive the standard size clamps 37 used in the medical profession. If the internal mesh ring 42 and attachment are manufactured as two pieces, the second diameter 44 of the internal mesh ring 42 is configured to receive and connect the attachment 48, also referred to as a gripper. The apparatus 40 can also be manufactured with the internal mesh ring 42 or the internal mesh ring and attachment 48.

The attachment can also have raised projections 50 or an elevated area to hold the medical device 37 in place.

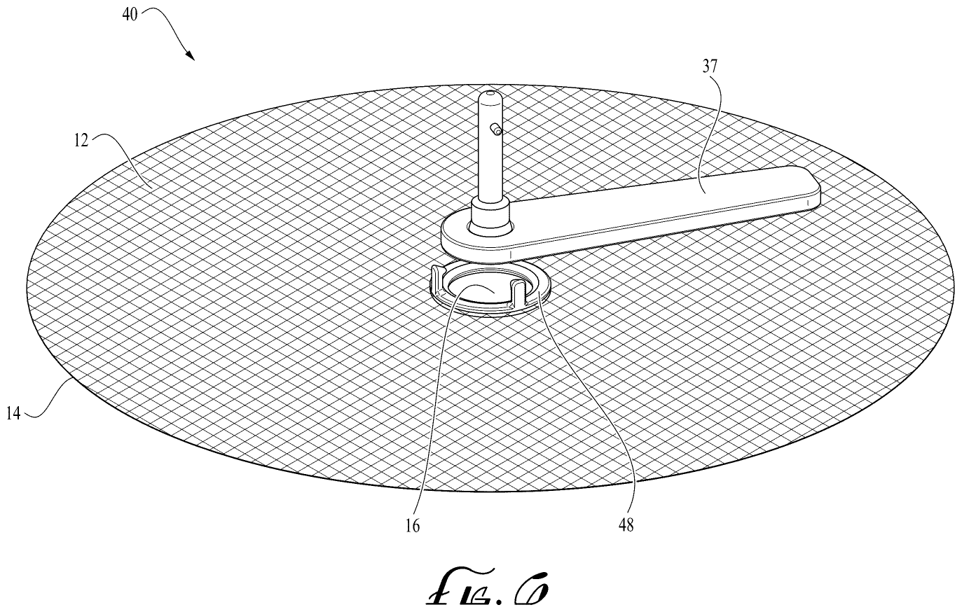

FIG. 5 is an exploded view of the apparatus 40 showing how the internal mesh ring 42 connects to the attachment 48 and the attachment receives the medical device 37.

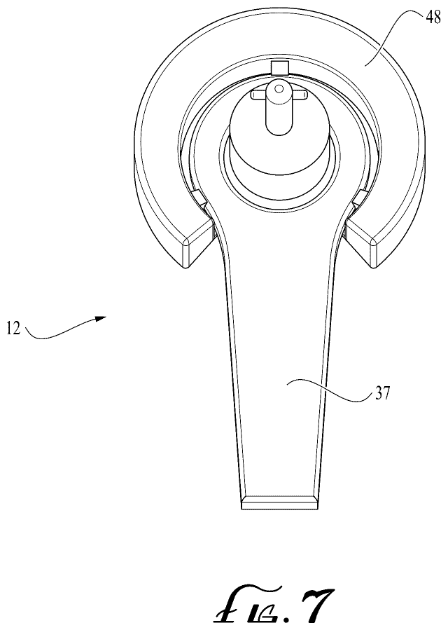

FIG. 6 is a perspective view of the apparatus attaching the medical device after the internal mesh ring 42 and attachment 48 have been secured to the body 12.

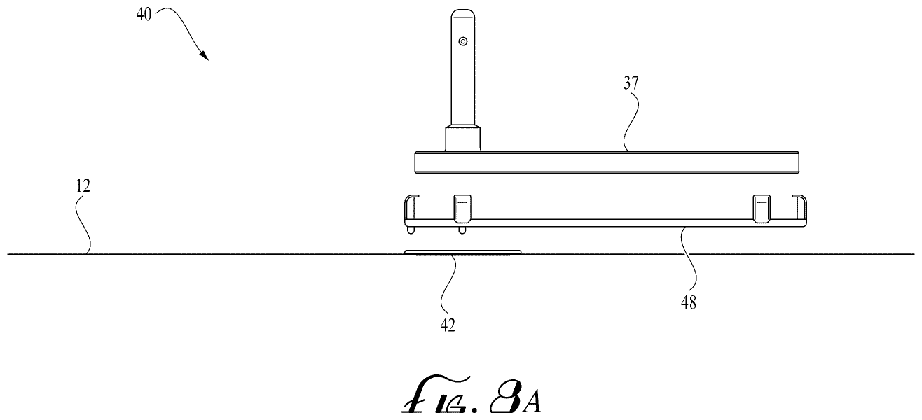

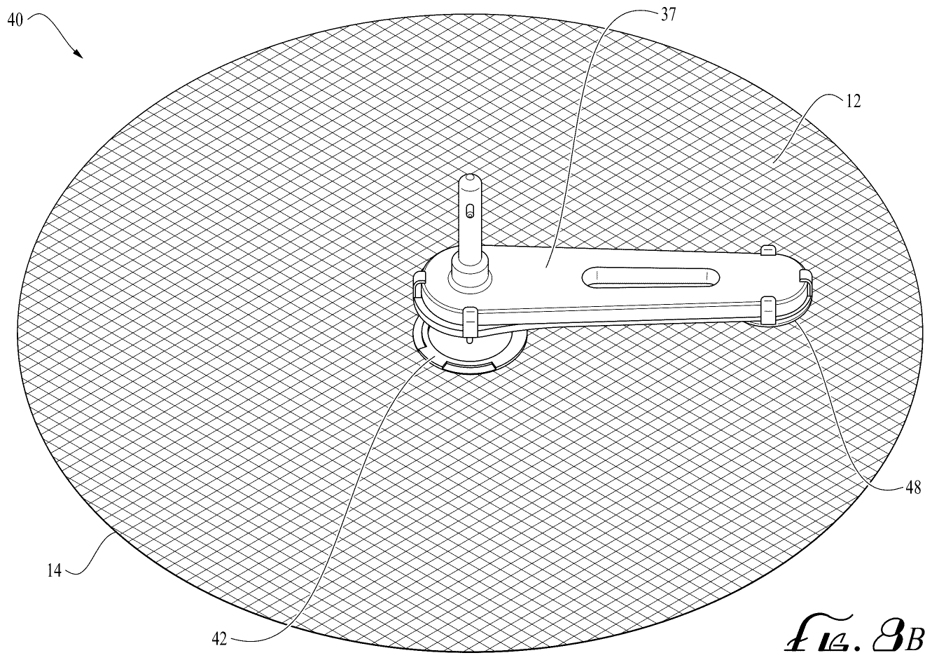

In FIG. 4 the attachment 48 is circular in cross-section, but it can be any shape including a semi-circle as shown in FIG. 7 or the shape of a clamp 37 as shown in FIGS. 8A and 8B. When the attachment 48 is in the shape of a clamp 37 it can assist the medical professional in securing the clamp 37 to the attachment 48.

In use, a medical professional selects the apparatus 40. If the apparatus has the internal mesh ring 42 and attachment 48 already connected the professional places the apparatus 40 over the penis of a patient generally on top of a sterile gown or drape, attaches the medical device 37 to the attachment 48, and performs the procedure. The attachment can be secured to the medical device with a snap fit. Alternatively, it can be locked in place with a twist lock such as shown in FIG. 8B. Similarly, the ring 42 can be attached to the attachment 48 with a snap fit.

If the apparatus 40 does not have the internal mesh ring 42 and attachment 48 already connected, the medical provider inserts the internal mesh ring 42 so that the second diameter 46 is projecting through the opening 16, secures an attachment 48 to the internal mesh ring 44, and attaches the medical device 37 to the attachment 48; or alternatively as shown in FIG. 8B, the attachment 48 can be attached to the medical device 37 and then connected to the internal mesh ring 42 prior to the procedure being performed.

Although this invention has been described with regard to preferred versions thereof, the invention is not limited to the preferred versions.

What is claimed is:

1. An apparatus for use during circumcision comprising a flexible mesh having an outer perimeter and a circular or oval opening therethrough, the opening being centrally or near centrally located, the opening having a diameter of from about 1 to about 1.5 inch, the outer perimeter of the mesh having a diameter of from about 6 to about 10 inches, the mesh being made of wires sufficiently close together that a scalpel pushed with a force of 20 Newtons cannot penetrate the mesh.

2. The apparatus of claim 1 wherein the mesh is a stainless steel mesh being made of wires about 0.5 to about 1.5 mm in diameter, the mesh being sufficiently flexible that the mesh can be doubled on itself.

3. The apparatus of claim 1 wherein the edges of the opening and the perimeter are sufficiently smooth to not injure or irritate a patient during circumcision.

4. The apparatus of claim 1 comprising a covering on the perimeter.

5. The apparatus of claim 4 wherein the opening has an edge, the apparatus comprising a covering on the edge of the opening.

6. The apparatus of claim 1 wherein the opening has an edge, the apparatus comprising a covering on the edge of the opening.

7. The apparatus of claim 1 wherein the opening is circular and the perimeter is circular.

8. A method for performing a circumcision on a patient comprising the steps of:
a) selecting an apparatus comprising a flexible mesh having an opening therethrough, the opening being at or near the center of the mesh;
b) placing a drape over an abdominal area of the patient, including over the femoral arteries of the patient;
c) placing the selected apparatus over the drape and over the patient's penis with the penis projecting through the opening and with the mesh proximate to the patient's abdominal area from penetration of the abdominal area during the circumcision; and
d) after step (c) circumcising the projecting penis.

9. The apparatus for use during circumcision, the apparatus comprising a body having an outer perimeter and a circular or oval opening therethrough, the opening being located in the body and centrally or located off center, the opening having a diameter of from about 1 to about 1.5 inch, the outer perimeter of the body having a diameter of from about 6 to about 10 inches, the body being formed of a flexible material and sufficiently resistant to penetration that a scalpel pushed with a force of 20 Newtons cannot penetrate the body.

10. An apparatus for use during a medical procedure involving a penis comprising a flexible body and having an outer perimeter and an opening therethrough, the opening having a diameter of from about 0.2 to about 1.5 inch, the outer perimeter of the body having a diameter of at least about 6 inches, the body being sufficiently strong that a number 10 scalpel pushed with a force of 10 Newtons cannot penetrate the body.

11. The apparatus of claim 10 wherein a number 10 scalpel pushed with a force of 20 Newtons cannot penetrate the body.

12. The apparatus of claim 11 wherein a number 10 scalpel pushed with a force of 50 Newtons cannot penetrate the body.

13. The apparatus of claim 10 wherein the body is sufficiently strong that a 28-gauge needle pushed with a force of 10 Newtons cannot penetrate the body.

14. An apparatus for use during a medical procedure on or proximate to a penis comprising a flexible body having an outer perimeter and an opening therethrough, the opening having a diameter of from about 0.2 to about 1.5 inch, the outer perimeter of the body having a diameter of at least about 6 inches, the body being sufficiently strong that a 28 gauge needle pushed with a force of 10 Newtons cannot penetrate the body.

15. The apparatus of claim 14 wherein the body is sufficiently strong that a 28-gauge needle pushed with a force of 50 Newtons cannot penetrate the body.

16. A method for performing a medical procedure on a patient comprising the steps of:
a) selecting an apparatus comprising a flexible body having an opening therethrough sized at a minimum that an infant penis having a circumference of 0.5 inch can project through the opening;
b) placing a drape over the abdominal area of the patent including the patient's femoral arteries;
c) placing the selected apparatus over the drape and over the patient's penis with the penis projecting through the opening and with the body proximate to the patient's skin for protecting the patient's abdominal area from penetration of the abdominal area during the medical procedure; and
d) after step (c) performing the medical procedure.

17. The method of claim 16 wherein the body is a mesh.

18. The method of claim 16 wherein the medical procedure is a circumcision.

19. An apparatus for use during a medical procedure on or proximate to a penis, the apparatus comprising a body with an opening therethrough, the opening sufficiently large that an infant penis having a circumference of 0.5 inch can project through the opening, the body being sufficiently strong that a number 10 scalpel pushed with a force of 10 Newtons cannot penetrate the body.

20. An apparatus for use during a medical procedure on or proximate to a penis, the apparatus comprising a body with an opening therethrough, the opening sufficiently large that an infant penis having a circumference of 0.5 inch can project through the opening, the body being sufficiently strong that a 28 gauge needle pushed with a force of 10 Newtons cannot penetrate the body.

21. The apparatus of claim 19 wherein the opening is centrally or near centrally located in the body.

22. The apparatus of claim 20 wherein the opening is centrally or near centrally located in the body.

23. The apparatus of claim 19 wherein the body has a diameter of at least six inches.

24. The apparatus of claim 20 wherein the body has a diameter of at least six inches.

25. An apparatus suitable for use during a medical procedure on or proximate to a penis comprising a flexible mesh having an outer perimeter and a circular or oval opening therethrough, the opening being in the body and being centrally located or located off center, the mesh being made of wires having a diameter of from about 0.02 mm to about 0.15 mm and the mesh has apertures sized from about 0.02 mm to about 20 mm.

26. An apparatus for use during a medical procedure involving a penis utilizing a circumcision device, the apparatus comprising:
(i) a flexible body and having an outer perimeter and an opening therethrough, the opening having a diameter of at least about 0.2 inch, the outer perimeter of the body having a diameter of at least about 6 inches; and
(ii) an attachment for removably securing the apparatus to the circumcision device.

27. The apparatus of claim 26 further comprising (iii) an interior mesh ring for attaching the attachment to the body, the interior mesh ring having a first diameter and a second diameter, wherein the first diameter is equal to greater than the diameter of the opening and the second diameter is less than the diameter of the first diameter

28. The apparatus of claim 27 wherein the attachment is a fastener at the opening and the circumcision device is a circumcision clamp.

29. A kit comprising the apparatus of claim 26 and a circumcision clamp.

30. A method for performing a medical procedure using a medical device on a patient comprising the steps of:
a) selecting an apparatus comprising:
(i) a flexible body having an opening therethrough sized at a minimum that an infant penis having a circumference of 0.5 inch can project through the opening,
(ii) an interior mesh ring having a first diameter and a second diameter, wherein the first diameter is equal to or greater than the diameter of the opening and the second diameter is less than the first diameter, and
(iii) an attachment for attaching the apparatus to the medical device;
b) placing a drape over the abdominal area of the patient including the femoral arteries;
c) placing the selected apparatus over the drape and over the patient's penis with the penis projecting through the opening and the interior mesh ring and with the body proximate to the patient's skin for protecting the patient's abdominal area from penetration during the medical procedure;
d) attaching the selected apparatus to the medical device with the flexible body clamped between the ring and the attachment; and
e) after step (d) performing the medical procedure.
